(19) United States
(12) Patent Application Publication
(10) Pub. No.: US 2011/0111485 A1
(43) Pub. Date: May 12, 2011

(54) SYSTEM AND PROCESS TO TREAT BIOMASS PILES WITH ENZYMES

(75) Inventor: Rodolfo ROMERO, Clifton Park, NY (US)

(73) Assignee: ANDRITZ INC., Glens Falls, NY (US)

(21) Appl. No.: 12/940,548

(22) Filed: Nov. 5, 2010

Related U.S. Application Data
(60) Provisional application No. 61/258,680, filed on Nov. 6, 2009.

(51) Int. Cl. C12S 3/04; C12M 1/40

(52) U.S. Cl. 435/277; 435/309.1

(57) ABSTRACT

A method for processing biomass including: conveying biomass to a pile of biomass on a platform; maintaining the biomass in the pile in a wetted condition, such that the biomass has a pH level in a predetermined pH range and a temperature in a predetermined temperature range; applying an enzyme to the biomass in the pile, wherein the enzyme extracts hemicelluloses from cellulosic fibrous material in the biomass; draining liquid from the pile of the biomass; removing the extracted hemicelluloses from the drained liquid, and returning to the pile at least a portion of the drained liquid after removal of the extracted hemicelluloses.

### CROSS RELATED APPLICATION

This application claims the benefit of application Ser. No. 61/258,680 filed Nov. 6, 2009, which is incorporated in its entirety by reference.

### BACKGROUND OF THE INVENTION

This invention relates to treating piles of biomass with enzymes and, particularly, relates to using a Ritter process to treat biomass piles with enzymes.

In a conventional Ritter process, cellulosic fiber, e.g., biomass, is mixed with water and discharged from a conveyor to form large piles of wetted cellulosic fibrous material. In these wetted piles, lactic acid bacteria consume residual sugars in the in cellulosic fiber to form organic acids. The bacteria inhibit other bacteria in the biomass that would otherwise prevent the fiber from rotting. As the wetted pile ages in a conventional Ritter process, the cellulosic fiber rots and becomes denser and more economical to handle.

Water and liquid filtrate are drained from the wetted piles, in a conventional Ritter process. The liquids are recirculated to the pile, such as by being sprayed onto the piles. The wetted piles are typically formed on platforms having channels for liquid drainage and filters to collect dirt and other solubles, such as phosphorus and potassium, from the drainage liquid. The dirt and solubles may be recovered from the drainage and used, for example, as fertilizer for the crop fields growing the biomass material that is later used to form the piles.

### BRIEF DESCRIPTION OF THE INVENTION

A method has been conceived and is disclosed herein for processing biomass comprising: conveying biomass to a pile of biomass on a platform; maintaining the biomass in the pile in a wetted condition, such that the biomass has a pH level in a predetermined pH range and a temperature in a predetermined temperature range; applying an enzyme to the biomass in the pile, wherein the enzyme extracts hemicelluloses from cellulosic fibrous material in the biomass; draining liquid from the pile of the biomass; removing the extracted hemicelluloses from the drained liquid, and returning to the pile at least a portion of the drained liquid after removal of the extracted hemicelluloses.

The predetermine pH range may be within a range of 3 to 5 pH, and the predetermined temperature range may be within a range of 70° F. to 122° F. (21° C. to 50° C.). The added enzyme may include one or more of the xylanases. Further, inorganic material may be removed from the drained liquid.

A method has been conceived and is disclosed herein for processing biomass comprising: conveying biomass to a pile of biomass on a platform; maintaining the biomass in the pile in a wetted condition; applying an enzyme to the biomass in the pile, wherein the enzyme extracts hemicelluloses from the biomass; draining liquid from the pile of the biomass; removing the extracted hemicelluloses from the drained liquid, and returning to the pile at least a portion of the drained liquid after removal of the extracted hemicelluloses.

The method may further comprise maintaining a pH of the pile in a range of 3 to 5 pH, and maintaining a temperature of the pile in a range of 70° F. to 122° F. (21° C. to 50° C.). The enzyme may include xylanases. Further, the method may remove inorganic material from the drained liquid. In addition, the extracted hemicelluloses may be removed by a separation process.

An apparatus has been conceived and is disclosed herein to process biomass comprising: a platform supporting a pile of biomass; a drainage channel in the platform adapted to receive drainage liquid from the pile of biomass; a circulation system for liquid drainage extracted from the pile through the drainage channel, the circulation system including one or more conduits receiving and transporting the liquid drainage to a liquid discharge device proximate to the pile; an enzyme source included in the circulation system and adapted to provide an enzyme to the liquid drainage to be discharged by the liquid discharge device.

The enzyme source may include a source of one or more xylanases. The circulation system may include: a separation stage which removes hemicellulosic material from the liquid drainage flowing through the conduits; a heat transfer device which maintains a temperature of the pile in a range of 70° F. to 122° F. (21° C. to 50° C.); a source of alkaline chemicals to be added to the drainage liquid flowing through the conduits, wherein the alkaline chemicals are added in an amount to maintain a pH of the pile in a range of 3 to 5 pH; a filtration device which separates the drainage liquid from particles in the drainage liquid flowing from the drainage channel, and a separation stage which extracts hemicellulosic material from the drainage liquid flowing through the conduits. The liquid discharge device may include a plurality of spray nozzles arranged above the pile.

### BRIEF DESCRIPTION OF THE DRAWINGS

FIG. 1 is a schematic diagram of a system for processing piles of biomass materials.

### DETAILED DESCRIPTION OF THE INVENTION

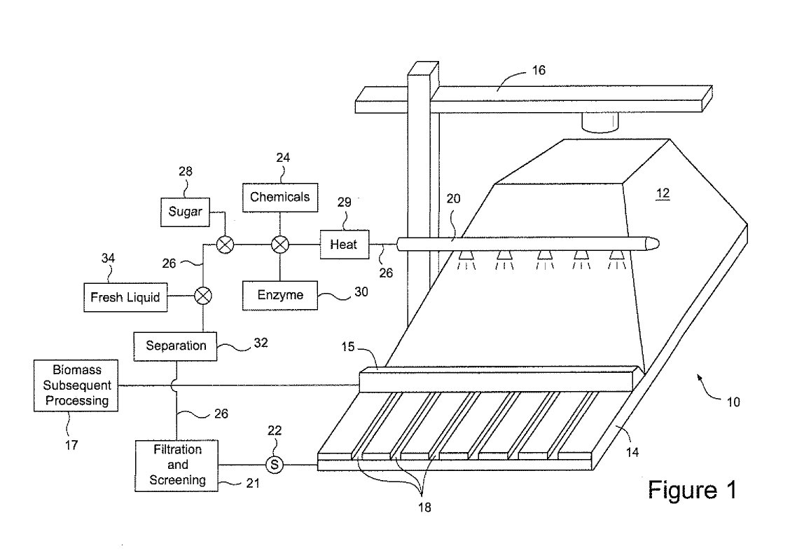

FIG. 1 is a schematic diagram of a mill storage system 10 that forms piles 12 of web biomass material, e.g., lingnocellulosic feedstock material, on a platform 14 below an overhanging biomass conveyor 16 which delivers the biomass material to the piles. After the biomass material has been processed in the piles, the processed material is removed by a lower biomass conveyor 15 arranged below or adjacent a lower edge of each pile 12. The piles 12 may be used to provide a constant source of biomass feedstock or other raw material for subsequent processes 17 to produce ethanol, such as fermentation, steam explosion and hydrolysis.

The platform 14 for the piles 12 includes drainage channels 18 to remove liquid from the bottom of the piles. The liquid removed from the bottom of the piles is returned to the top of the pile by, for example, a liquid spray system 20. During the drainage process, non-lignocellulosic material is removed from the drained liquid such as by filtration and screening 21.

The mill storage system 10 may be operated to process the biomass material pile 12 using a Ritter process. In a conventional Ritter process, the pH levels of large piles of wet biomass material are controlled by draining liquids from beneath the piles and retuning the drainage liquid to the piles 12, such as to the top of the piles.

To control the pH of the biomass in the piles 12, the pH of the liquid drained from the pile is monitored, such as by pH sensors 22, and adjusted in a circulation system which adds chemicals 24, e.g., ash and other alkaline chemicals, to the circulation conduit 26 through which flows the liquid being returned to the top of the piles.

The pH of the piles is typically maintained at between 3 to 5, and is preferably maintained below a pH of 4.2. Maintaining the pH in the desired ranges prevents or suppresses the growth of unwanted bacteria and fungi in the piles. If not prevented or suppressed, the unwanted bacteria may damage the fibers and carbohydrates in the biomass.

Preferably, the internal temperature of the biomass in the piles remains relatively constant in a range of 70 degrees Fahrenheit (70° F.) to 122° F. (21 degrees Celsius (21° C.) to 50° C.). The flow of returned drainage liquid may be heated in a heat exchanger 29 in the circulation system to control the temperature of the piles.

Preferably, anaerobic conditions are maintained in the piles 12. Anaerobic conditions are maintained in the pile by feeding bacteria in the biomass with naturally present sugars in the biomass or by adding sugars to the piles, such as by introducing sugars 28 to the conduit 26 for the returning liquid. Anerobic conditions in the biomass assist in suppressing attacks by organisms, e.g., bacteria, on carbohydrates and fibers in the biomass. This process allows for concentration of desired material, e.g., hollocellulose in the biomass.

The yield of glucose from the piles 12 of biomass can be enhanced by reducing the amount of hemicelluloses in the piles. Hemicellulose is primarily in the form of xylan for most usable biomass feed stocks. Enzymes are added to the piles 12 to break down the hemicelluloses in the biomass. The enzymes may be added to the flow of drainage liquid flowing through the conduits 26 of the circulation system.

The piles are maintained under controlled biological and anaerobic conditions, such as is provided in a Ritter Process. Enzymes 30, such as xylanases, are added to the biomass in the piles to break down the hemicelluloses and thereby increase the reactivity of the cellulose in the biomass. The enzymes may be added from an enzyme source 30 that introduces the enzyme to the flow of returned liquid in the conduit 26 leading to the spray system 20. Biomass with increased reactivity is useful for subsequent processing, such as by sacharification and fermentation processing used in the production of ethanol fuel.

By adding enzymes 30 to and heating 29 the liquid in conduit 26 being returned to the piles, a desired amount of enzymes are maintained in the biomass of the piles. In addition, the temperature of the piles is preferably controlled such that the biomass is in a temperature range of 70° F. to 122° F. (21° C. to 50° C.) and the pH of the biomass is in a range of 3 pH to 5 pH, e.g., at 4.2 pH. When the piles 12 are maintained under these conditions, the enzymes in the biomass will extract the hemicelluloses (C5 sugars) from biomass and the extracted hemicelluloses flows with the liquid that flows through the piles and into the drainage channels 18 of the platform 14.

The drainage liquid withdrawn from the piles through the drainage channels flow is split or otherwise processed to capture the hemicellulose material extracted from the piles 12 and flowing in the drainage liquid through the conduits 26. The hemicellulose may be removed by a conventional separation stage 32 in the flow of the drainage liquid removed from the piles. In addition, a filter or separator 21 in the circulation system removes inorganic material from the liquid to be returned to the pile.

After the hemicelluloses and inorganic material are removed, the liquid, or a portion thereof, flows through conduit 26 and is returned to the pile. A source of fresh liquid, e.g., water, 34 introduces the fresh liquid to the conduit 26 returning liquid to the pile.

By introducing enzymes into the piles 12 and controlling the temperature and pH of the piles, in the manner described herein, the biomass produced from the piles provides cellulose having increased digestibility, which may provide for reductions enzyme dosage of the cellulose in a sacharfication vessel. Additional potential benefits of the processes described herein include a reduction in the amount of ash needed to be added to the biomass in the piles, a reduction in the fire hazard of the biomass in the piles and elsewhere stored, and enhanced throughput of the biomass by concentrating the desired biomass material to have a higher concentration of the desired hollocellulose.

By concentrating the hollocellulose in the biomass material from the piles, the biomass processing equipment downstream of the piles need only have sufficient capacity and energy consumption to process the concentrated biomass. The capacities and energy consumption of the equipment may be less than that of equipment needed to process biomass that had not been concentrated. In addition, the removal of inorganic material from the liquid extracted from the piles allows for the inorganic material, e.g., minerals, to be returned to the fields that grow the biomass and, thus, for, improved sustainability of the harvestable area of those fields.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

I claim:

1. A method for processing biomass comprising:
conveying biomass to a pile of biomass on a platform;
maintaining the biomass in the pile in a wetted condition, such that the biomass has a pH level in a predetermined pH range and a temperature in a predetermined temperature range;
applying an enzyme to the biomass in the pile, wherein the enzyme extracts hemicelluloses from the biomass;
draining liquid from the pile of the biomass;
removing the extracted hemicelluloses from the drained liquid, and
returning to the pile at least a portion of the drained liquid after removal of the extracted hemicelluloses.

2. The method of claim 1 wherein the predetermine pH range is within a range of 3 to 5 pH, and the predetermined temperature range is within a range of 70° F. to 122° F. (21° C. to 50° C.).

3. The method of claim 1 wherein the enzyme includes xylanases.

4. The method of claim 1 wherein inorganic material is removed from the drained liquid.

5. The method of claim 1 wherein the extracted hemicelluloses is removed by a separation process.

6. A method for processing biomass comprising:
conveying biomass to a pile of biomass on a platform;
maintaining the biomass in the pile in a wetted condition;
applying an enzyme to the biomass in the pile, wherein the enzyme extracts hemicelluloses from the biomass;
draining liquid from the pile of the biomass;
removing the extracted hemicelluloses from the drained liquid, and
returning to the pile at least a portion of the drained liquid after removal of the extracted hemicelluloses.

7. The method of claim 6 further comprising maintaining a pH of the pile in a range of 3 to 5 pH.

8. The method of claim 6 further comprising maintaining a temperature of the pile in a range of 70° F. to 122° F. (21° C. to 50° C.).

9. The method claim 6 wherein the enzyme includes xylanases.

10. The method of claim 6 wherein inorganic material is removed from the drained liquid.

11. The method of claim 6 wherein the extracted hemicelluloses is removed by a separation process.

12. An apparatus to process biomass comprising:
a platform supporting a pile of biomass;
a drainage channel in the platform adapted to receive drainage liquid from the pile of biomass;
a circulation system for liquid drainage extracted from the pile through the drainage channel, the circulation system including one or more conduits receiving and transporting the liquid drainage to a liquid discharge device proximate to the pile;
an enzyme source included in the circulation system and adapted to provide an enzyme to the liquid drainage to be discharged by the liquid discharge device.

13. The apparatus of claim 12 wherein the enzyme source includes a source of one or more xylanases.

14. The apparatus of claim 12 wherein the circulation system includes a separation stage which removes hemicellulosic material from the liquid drainage flowing through the conduits.

15. The apparatus of claim 12 wherein the circulation system includes a heat transfer device which maintains a temperature of the pile in a range of 70° F. to 122° F. (21° C. to 50° C.).

16. The apparatus of claim 12 wherein the circulation system includes a source of alkaline chemicals to be added to the drainage liquid flowing through the conduits, wherein the alkaline chemicals are added in an amount to maintain a pH of the pile in a range of 3 to 5 pH.

17. The apparatus of claim 12 wherein the liquid discharge device includes a plurality of spray nozzles arranged above the pile.

18. The apparatus of claim 12 wherein the circulation system includes a filtration device which separates the drainage liquid from particles in the drainage liquid flowing from the drainage channel.

19. The apparatus of claim 12 wherein the circulation system includes a separation stage which extracts hemicellulosic material from the drainage liquid flowing through the conduits.
